(19) United States
(12) Patent Application Publication
(10) Pub. No.: US 2011/0171732 A1
(43) Pub. Date: Jul. 14, 2011

(54) HIGHLY POROUS SOLID MATERIAL MADE OF BIODEGRADABLE POLYMER AND METHOD OF FABRICATING, PROCESSING, AND CELL-SEEDING THE SAME

(76) Inventors: Corrado Mandoli, Ibaraki (JP); Enrico Traversa, Ibaraki (JP)

(21) Appl. No.: 12/916,919

(22) Filed: Nov. 1, 2010

(30) Foreign Application Priority Data
Jan. 12, 2010 (JP) 2010-003539

(51) Int. Cl. C12N 5/07; F25D 25/00; C08J 9/26

(52) U.S. Cl. 435/395; 62/62; 264/49

(57) ABSTRACT

[Problems] A purpose of the invention is to provide highly porous 3D scaffolds made of biodegradable polymer such as poly-lactic acid, which can be preferably used for cell culture.

Another purpose of this invention is to provide a method for seeding cells homogeneously inside the above mentioned 3D highly porous scaffolds.

Another purpose of this invention is to provide a top-down processing method for obtaining planar porous 2D scaffolds starting the above mentioned highly porous scaffolds to be used as building blocks for thick tissue reconstruction.

[Solution] Highly porous scaffolds made of biodegradable polymer such as poly-lactic acid can be fabricated by following steps:
(i) biodegradable polymer is dissolved in dioxane at the defined concentration;
(ii) a vessel containing the solution is mounted on a metal plate thermally driven by a cold finger to a defined temperature below 10° C., and then the vessel is kept on the plate, until the solution undergoes complete phase separation; and
(iii) the obtained solids are immersed into an alcoholic aqueous solution to leach out the dioxane.

### FILED OF INVENTION

This invention relates to morphologically new highly porous solid materials made of biodegradable polymer whose pore network is characterized by the ordered repetition of parallel closely-packed dendritic-like cavities. The invented highly porous solid materials are preferably used for cell culture as three dimensional (3D) scaffolds.

This invention also relates to (i) a fabrication method for the above highly porous solid materials, (ii) a method for seeding cells homogeneously within the 3D scaffold, (iii) a method for obtaining bi-dimensional (2D) scaffolds starting from the 3D construct, to be used as stacking planes for thick tissue reconstruction.

### BACKGROUND ART

Regenerative medicine has become a major modern therapeutic approach for the rescue of those diseases that can be treated only by organ transplantation, due to the shortage of organ donors. Despite the encouraging results obtained in reconstructing hard tissues, such as bone and cartilage, and flat soft tissues, such as cornea and skin, one of the main unresolved problems of tissue engineering (TE) is the difficulty in reconstructing healthy thick tissues. A number of TE strategies have been proposed in literature to overcome this limitation. Among them, a promising approach comes from the use of biodegradable three-dimensional constructs (3D scaffolds). An ideal 3D scaffold should incorporate the information able to trigger the specific tissue reproduction and should guarantee at the same time perfusion, vascularization, growth factor delivery, and all those processes responsible for cellular long term retention.

With respect to soft tissue repair using 3D scaffolds, poly-lactide acid (PLA) is among the most widely studied biomaterials and lactide-based scaffolds represent today the main portion of the worldwide bio-support industry. PLA degradation kinetics is well known and can be tuned by modifying the molecular weight or the chemical composition.

The hydrophobic nature of PLA can favor cellular adhesion, but simultaneously represents a massive hurdle for cell accommodation into 3D scaffolds. A variety of post-treatments to modify the PLA hydrophobicity and roughness have been proposed in the literature (non-patent literature 1). These methods, although effective for increasing cell adhesion in 2D scaffolds, have the drawback to deeply modify the 3D scaffold architecture by, for example, causing partial erosion in the architecture, with loss of important morphological features. Due to its watery nature, cell culture medium cannot spontaneously permeate the hydrophobic PLA scaffolds. Moreover, cell migration is also hindered by the capillary forces exerted by scaffold porosity. The fabrication of highly porous scaffolds with non-tortuous, interconnected pores of a size allowing cell mobility can be a solution to such problems.

The architecture of these scaffolds can be also effective in permitting nutrient perfusion, which is the main limiting factor for growing in vivo healthy thick tissues on 3D scaffolds. The lack of perfusion into the core regions results in poor colonization and cells confinement on the scaffold cortical layers. The main problems of the 3D scaffolds are therefore the lack of a suitable pore arrangement able to facilitate diffusion of nutrient and oxygen and to allow cell migration deep into the scaffold, together with the inability to host more organized structures like blood vessels. The pore architecture can be designed by modifying the processing methods. A large number of processing techniques have been reported for PLA, including gas foaming, particulate leaching, fiber bonding, electrospinning, soft-lithography, and phase separation. Lactide-based scaffolds made by thermally induced phase separation (TIPS) have demonstrated their suitability to host cell systems since their multi-scale porosity could be able to support cell-matrix interaction at ideally any scale level (non-patent literature 1).

TIPS is basically a three-step process in which a homogeneous polymer-solvent solution is brought to phase separate upon the application of a thermal gradient into a polymer-rich phase and a solvent-rich phase. Porosity is finally obtained by leaching out the solvent phase by exposing to a third agent that acts as a non-solvent for the polymer. A number of ternary systems have been investigated for producing polymeric structures by TIPS. In particular, for PLA-based systems, the use of dioxane as the polymer solvent turned out to be the election choice, due to its relatively high melting temperature (T=11.8° C.) and ease of removal. Moreover, it has been demonstrated that dioxane can crystallize into different geometries depending on its concentration within the polymer solution and quenching conditions (non-patent literature 1). The ability to control solvent shaping under solidification is crucial because crystallite geometry and order act as a template for porosity. Imposing an anisotropic thermal gradient to the polymeric solution can force dioxane crystallization along specific directions, ordering the whole system at a macroscopic level (non-patent literature 2). Several research groups managed to obtain densely packed arrays of parallel micro channels by applying uni-directional TIPS to mixed PLA and poly(L-lactic-co-glycolic acid) (PLGA) solutions in dioxane (non-patent literature 3-5). However, this configuration may represent an obstacle for intense cell-to-cell signaling between adjacent channels, or even result in shortening of the lifetime of cells in the case of inadequate nutrient supply/diffusion through the micro-cavities of the inner walls. In addition, constraint of growth/migration of cells only to one specific direction may impede cells arrangement into more complex structures or prevent vessels capillary sprouting. Another problem which constantly affects the 3D scaffold, resides in the difficulty for cells to entirely colonize the scaffold due to inefficient cell seeding. This problem could be circumvented by using more efficient seeding techniques or by constructing 3D tissues using stacks of 2D seeded scaffolds.

### OUTLINE OF INVENTION

### Problems to be Solved by Invention

A purpose of the invention is to provide highly porous 3D scaffolds made of biodegradable polymer such as poly-lactic acid, which can be preferably used for cell culture.

Another purpose of this invention is to provide a fabrication method for the above highly porous 3D scaffolds.

Another purpose of this invention is to provide a method for seeding cells homogeneously inside the above mentioned 3D highly porous scaffolds.

Another purpose of this invention is to provide a top-down processing method for obtaining planar porous 2D scaffolds starting the above mentioned highly porous scaffolds to be used as building blocks for thick tissue reconstruction.

### Means for Solving Problems

Along these considerations, we have set up a protocol for scaffold fabrication of specific morphology in a reproducible manner by using directional thermally induced phase separation (dTIPS). The set of conditions identified corresponds to the porosity arrangement that would better support diffusive phenomena as well as suit blood vessels accommodation, thus easing angiogenesis promotion.

Namely, this invention provides highly porous solid materials (or scaffolds) made of biodegradable polymer whose pore network is characterized by the ordered repetition of parallel closely-packed dendritic-like cavities.

This invention also provides a fabrication method for highly-porous solid materials (or scaffolds) made of biodegradable polymer whose pore network is characterized by the ordered repetition of parallel closely-packed dendritic-like cavities. The method comprises the following steps:

(i) biodegradable polymer is dissolved in dioxane at the defined concentration (“a step of dissolving biodegradable polymer”);
(ii) a vessel containing the solution is mounted on a metal plate thermally driven by a cooling unit (or cold finger) to a defined temperature below 10° C., and then the vessel is kept on the plate, until the solution undergoes complete phase separation. In other words, dioxane separates from the polymer and freezes in ordered manner along the cooling direction imposed to the solution, in form of parallel closely-packed dendriforms. At the same time, the dissolved biodegradable polymer is expelled and solidifies around the dioxane dendrite-like crystals. (“a step of directional thermally induced phase separation”); and
(iii) the obtained solids are immersed into an alcoholic aqueous solution to leach out the dioxane (“a step of removal of dioxane”).

The invention also provides a novel vacuum-assisted cell seeding method for allowing instant and deep cellular colonization of the 3D scaffold. The method was developed ad hoc for the constructs of the present invention and takes advantage of the low tortuosity and high mass transport of the scaffolds porosity. The method includes the following steps:

(i) the scaffold is positioned in a syringe and preliminarily undergoes a vacuum treatment aimed to replace the air trapped within the porous cavities with cell-free culture medium. In fact, trapped air can hinder homogeneous cell infiltration deep in the entire scaffold volume.
(ii) the flooded scaffold is positioned in another syringe and undergoes a vacuum treatment aimed to replace the cell-free culture medium with fresh cell-loaded medium.
(iii) the scaffold containing cell-loaded medium is placed in culture well, covered with fresh medium and kept in incubator.

In addition, the invention also provides a top-down method for obtaining 2D planar porous scaffold of biodegradable polymer having precisely oriented pore geometry, starting from the 3D dTIPS scaffolds of the present invention. The method requires the 3D scaffold to be embedded with specific agent and smart cut by means of cryostat. Once thin slices are produced, they are cell cultured and stacked to build up a 3D tissue (bottom-up approach). The method for obtaining 2D porous scaffolds from a 3D dTIPS scaffold includes the following steps:

(i) the scaffold is positioned in a syringe and undergoes a vacuum treatment aimed to replace the air trapped within the porous cavities with a suitable bio-compatible, bio-resorbable embedding agent. The embedding agent is selected among those used for improving cell adhesion and/or proliferation, and/or inducing cell differentiation, and/or exerting beneficial effects on the cell culture.
(ii) the flooded scaffold is exposed to low temperature to allow solidification of the embedding agent.
(iii) the embedded scaffold is mounted on a cryostat and sliced along direction parallel to the channel orientation, so that precisely oriented sections are produced. Moreover, the resulting 2D scaffolds are already coated with the embedding agent which improves cell adhesion and/or proliferation, and/or induces cell differentiation, and/or exerts beneficial effects on the cell culture.

### EFFECTS OF INVENTION

The invented highly porous biodegradable solid materials (or scaffolds) has a new morphology (pore/channel network) which is characterized by the ordered repetition of parallel closely-packed dendrite-like cavities, resembling the vascular patterns. The original seeding method specifically developed for these scaffolds allows instant and efficient cellular colonization of the construct, which is unachievable in scaffolds with differently organized porosities. The engineered scaffolds of the present invention are expected to serve for application aimed to vascular net and angiogenesis.

According to the invented fabrication and biological methods, we can easily produce the above highly porous biodegradable scaffolds and successfully culture them in vitro.

### BRIEF DESCRIPTION OF DRAWINGS

FIG. 1 Left: schematic sectional view of an apparatus for the fabrication. Right: whole view of “a cold finger”.

FIG. 2 SEM images of scaffolds obtained in Example 1 by directional TIPS carried out at −40° C. onto 6.4% PLLA in 1,4 dioxane solutions: (left to right, starting from the left upper corner) cross sectional view (a), top view of a PLLA scaffold prepared by dTIPS, 4.5 cm in diameter and 0.6 mm thick showing the circular pore arrangement observable at the macro-scale (b), top (c) and back (d) zoomed views of surface morphology resulting from the protrusion of the lamellae on the surface.

FIG. 3 Schematic sectional view of single dendrite-like solid of 1,4-dioxane obtained by freezing from the bottom of a mold.

FIG. 4 (a) Exploded view of the schematics of the scaffold; (b) SEM visualization of a surface terminating single PLLA dendritic unit obtained by dTIPS; (c) Schematics of a multi layered segment of the unit: growth (z), in-row (y) and row-to-row (x) directions are indicated together with the 20°-45° lamellar bending.

FIG. 5 Schematics of the vacuum-aided deep cell seeding method proposed: (a), (b) and (c) represent scaffold positioning and locking phase, aspiration phase, and expulsion phase, respectively.

FIG. 6 Cross sectional view of a scaffold made by dTIPS. (a) before cell seeding, and (b) 18 h after cell seeding in Example 2. Images were acquired under optical microscope at different magnifications, after hematoxylin/eosin cell staining.

FIG. 7 Schematics of cellular infiltration mechanisms upon the application of a pressure gradient: (a) top surface funneled inlet (side to main channel flow), (b) back surface reverse funneled inlet (main to side channels flow)

FIG. 8 2D construct obtained from smart cut of 3D dTIPS scaffold by means of a cryostat, (a) at high and (b) low magnification. The construct corresponds to a single dendritic plane as defined in FIG. 4a. The images were acquired under optical microscope, respectively. (c) fluorescence image of the single PLLA 2D strip after 3 DIV of MSC culturing. (d) Confluent MSC proto-tissue grown over a two layered scaffold. The construct was made by bottom-up fabrication, by the superposition of two 2D dTIPS scaffolds previously cultured for 15 DIV.

FIG. 9 Schematics of the proposed bottom-up approach for the fabrication of healthy thick tissues. The term “core scaffold” was used to indicate the vascularized 2D dTIPS scaffold.

### MODE OF IMPLEMENTING INVENTION

At first, we explain a fabrication method for the invented highly-porous solid materials (or scaffolds). Here, “highly-porous” solid materials mean ones exhibiting overall porosities of 80% to 99% (more preferably 88% to 96%) as determined by porosity measurements as described below (in Example).

As we said above, the fabrication method comprises the following steps (i)-(iii):

(i) a step of dissolving biodegradable polymer;
(ii) a step of directional thermally induced phase separation; and
(iii) a step of removing dioxane.

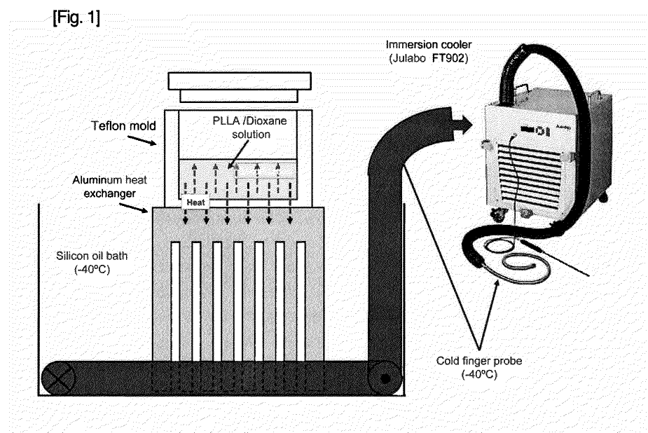

Regarding an apparatus for the fabrication, we use a cooling unit, oil bath, a thermally driven metal plate, and a vessel for the solution. FIG. 1 (left) shows a schematic sectional view of an apparatus for the fabrication, and FIG. 1 (right) shows whole view of “a cold finger” as a cooling unit.

Here, as a vessel for the solution we can use preferably a poly-tetrafluoroethylene mold, because of its inertness towards the solvents involved in the process as well its low thermal conductivity (2.700E-03 W/cmK) in the temperature range investigated, which ensures the thermal flux to be mainly localized at the bottom of the mold (which is in direct contact with the aluminum cold plate), and minimized at the mold walls. In this way, dioxane solidification is forced to occur vertically within the solution (i.e from bottom to top), with a negligible horizontal component.

The solution containing vessel can be fabricated in order to assume any shape, in principle. The geometry can be chosen from cubic, cylindrical, etc. However, the cylindric-type shape is preferable due the absence of edges and corners (e.g. cubic shaped mold) where border effects can manifest, affecting crystallites growth and geometry, and altering the ordered configuration. Moreover, cylindrical vessels are easier to fabricate. The size of the vessel can be chosen from a wide interval depending on the purpose. The typical size adopted in this work (expressed by the vessel cross sectional area) varied from about 20 cm² to 50 cm² (corresponding to diameters of about 5 cm to 8 cm, respectively). Lower sizes are not advisable, since the border effects at the vessel walls can become considerable respect to the total volume of the solution. On the other side, larger vessels would require the use of higher amounts of polymer to achieve same scaffold thickness, with an increase of experimental costs. Moreover, as the area interested by the thermal flux is increased, the possibility of inhomogeneous thermal conditions across the solution is also increased.

As the raw polymeric material for fabricating the scaffolds, we can use biodegradable, thermoplastic, aliphatic polyesters, and their copolymers. The aliphatic polyester polymers include poly(L-lactide) (PLLA), which is the naturally occurring optical isomer of polylactide (PLA), and poly (D,L-lactide) (PDLA), which is the synthetic blend of the two PLA isomers D-lactide and L-lactide.

The aliphatic polyester copolymers can comprise poly (D,L-lactide-co-glycolide) (PLGA), in which lactide to glycolide molar ratios can vary from 50:50 to 90:10 (e.g., PLGA 65:35), poly(L-lactide-co-glycolide-co-caprolactone) (PLGC), in which lactide molar ratio can vary from 60% to 80%. and Poly(DL-lactide-co-caprolactone), in which lactide molar ratio can vary from 30% to 90%.

Among them, we can use PLLA most preferably. With respect to soft and hard tissue repair, PLLA has always been one of the most utilized biomaterials (non-patent literature 6-9). Due to its high affinity towards cellular systems, chemical and mechanical stability, PLLA is currently used in several FDA approved biomedical and foodservice applications (non-patent literature 9). PLLA biodegradation kinetics in vivo, in the environment and in compost are well known and can be tuned by modifying the molecular weight or the chemical composition. Finally, high molecular weight PLLA is rarely effected by fungi, mold, or other microbes at ordinary temperatures.

Respect to PLGA and PLGC, PLLA is much stiffer, its degradation and resorption processes are slower, and accompanied by a higher inflammatory response. However, as the scaffold is required to be highly hydrophobic for enhancing cell adhesion, and mechanically self-sustainable, even in the biological environments, PLLA was generally preferred in the examples below to both PLGA and PLGC for scaffold fabrication. The choice is also justified by the much lower price of PLLA respect to both PLGA and PLGC.

As dioxane (or solvent) we can use 1,4-dioxane, 1,3-dioxane or 1,2-dioxane. Among them, we can use 1,4-dioxane (melting point: 11.8° C.) preferably, because it assumes a favorable crystal shape of crystals below its solidification temperature (11.8° C.), and then it can be easily removed by alcohol aqueous solution without loss of PLLA (or biodegradable polymer) nor any alteration of the constituted PLLA architecture.

When we use PLLA as biodegradable polymer and 1,4-dioxane as solvent, a concentration of PLLA in the range of 2 wt % to 15 wt % can be chosen. However, a PLLA concentration in the range of 4 wt % to 7 wt % is preferable in order to obtain aimed highly porous solid materials (or scaffolds) applicable to cell cultures.

In the step (ii) (step of directional thermally induced phase separation), as “the defined cold temperature” we can preferably use a constant temperature in between −20 and −80° C. Choosing this temperature condition can bring a directional thermal induction from bottom to top. As a result, dissolved dioxane freezes in the repeated order of parallel closely-packed dendriform by directional thermally induced phase separation and then dissolved biodegradable polymer solidifies.

After step (ii) the obtained solids are immersed into an alcohol/water solution to leach out (or remove) the dioxane. As alcohol we can use ethanol or methanol, and its concentration is preferable in the range of 50 wt % to 90 wt %. Thus we can obtain the aimed highly porous solid materials (or scaffolds).

The direction of the main pores (or channels/holes) of the obtained materials (or scaffolds) is generally perpendicular from bottom to top.

Here, the diameter of the main pores of the dendric-like cavities is preferably 70±10 μm and that of the side branches is preferably 45±5 μm respectively. The distance between adjacent main pores is preferably 80±20 μm.

The side branches generally depart from the main pores at the angle of 45° to 70° with respect to the main pore axis.

### >The Reason why Morphologically New Highly Porous Solid Materials can be Obtained>

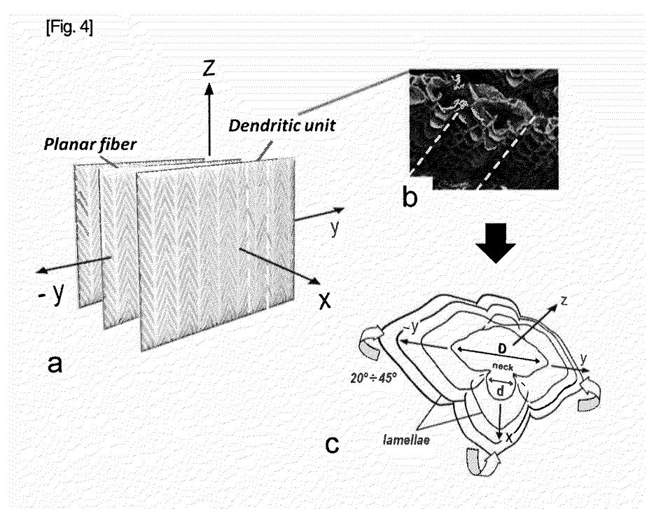

Forcing the thermal gradient to only occur in the perpendicular direction as shown in FIG. 1 results in the formation of densely packed vertical arrays of dioxane crystals (FIG. 3 shows schematic representation of single dendrite-like solid of dioxane obtained by freezing from the bottom of a mold.). After the sample (biodegradable polymer-dissolved dioxane solution) reached the thermal equilibrium at e.g. −40° C., the dioxane crystals are then dissolved in an EtOH/H₂O bath at e.g. −18° C., and their fingerprints are left in the polymeric matrix. The processing is reliable and reproducible. The resulting porosity is characterized by a long-range ordered hierarchical structure.

With respect to the 2D dTIPS supports fabrication by means of cryostat: as the biocompatible, biodegradable embedding agent we made use of ultrapure water-based or PBS-based solutions, in which one or more of the following components was dissolved: glycosaminoglycans (GAGs) including proteoglycans, such as heparan sulfate, keratan sulfate, chondroitin sulfate, and non-proteoglycan, such as hyaluronic acid; disaccharides, such as sucrose, etc.; extracellular matrix constituting proteins, such as fibronectins, collagens, elastins, laminins, and others. Bovine derived, porcine derived, or fish derived gelatins; finally, poly-l-lysine solutions can be used. The concentration of each of the mentioned components ranged from 0.1% to 40%, depending on the water solubility of the single components. In one preferred embodiment, the component (agent) is sucrose in ultrapure water solution, and the concentration of sucrose ranges from 1% to 40% in H₂O. In another preferred embodiment, the component is bovine derived gelatin in ultrapure water, and the concentration of bovine derived gelatin ranges from 1% to 40% in H₂O. In another preferred embodiment, the agent is bovine derived gelatin in PBS, and the bovine derived gelatin solution is used at a concentration of 0.1% to 30% in PBS.

### EXAMPLES

### Example 1

### (1) Scaffold Synthesis

Commercial PLLA (Mwa=100,000-150,000, Aldrich, USA) was dissolved in 1,4-dioxane (Aldrich, USA) at the fixed concentration of 6.4 wt %. 10 ml of the solution was cast into a circular poly-tetrafluoroethlene mold (inner diameter: 45 mm, inner depth: 30 mm), and the solution-containing mold was mounted onto a cold finger (FIG. 1) thermally driven Al plate set at −40° C. (the temperature to be assumed that the resulting channel size in the scaffolds would be large enough to accommodate cells) and thus the thermal gradient was applied for 10 hours (time enough to reach the thermal equilibrium at −40° C.) from the bottom to the top of the solution free surface, in the perpendicular direction. After solidification, the samples were immersed into 100 ml of an 80 wt % ethanol aqueous solution to leach out the solvent or 1,4-dioxane. The only parameter affecting the speed of the dioxane extraction process is the 80 wt % ethanol bath temperature; too high extracting temperature, e.g. room temperature, may result in dioxane melting with localized polymer redissolution and loss of the pre-formed scaffold architecture. To avoid an excessively slow process while still preserving scaffold features, the extraction temperature was initially set at −18° C. for two days and then gradually brought to room temperature. Ethanol solution was finally removed by vacuum drying the scaffold for 5 h at room temperature. The as-made constructs were cut into round samples of 14 mm in diameter, using a multi-shaped hollow-punch, and then stored in a desiccator until use.

### (2) Estimation

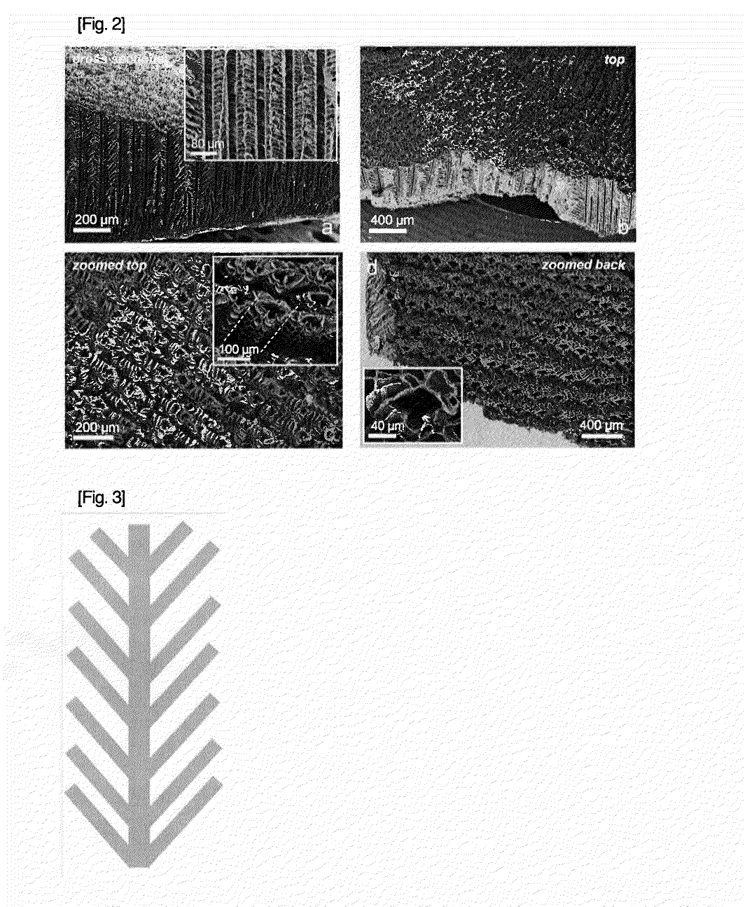

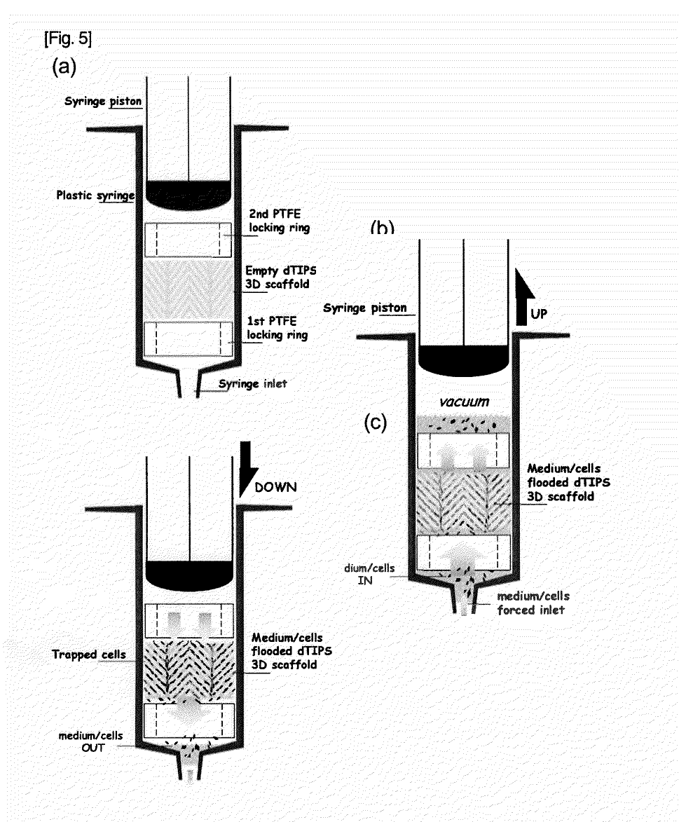

FIG. 2 shows the SEM images of the morphology of the obtained scaffolds. Starting from the cross sectional view (FIG. 2(a)), we can observe an array of straight parallel channels of about 20 μm diameter (inset 2(a)) going across the entire scaffold thickness. Each channel exhibits side tubular branches of comparable diameter departing from the whole channel length at about 45° to 70° with respect to the channel axis. As a result, the observed cross sections assume a typical multiple fishbone-like pore arrangement, as depicted in the figure. The holes in FIG. 2 correspond to the top (FIG. 2(b) and FIG. 2(c)) and back (FIG. 2(d)) surface channel terminations. As visible, the channel section, which is the void area with dimensions d and D shown around the center of FIG. 4(c), is characterized by a three-poled stingray-like shaped section. The pores are arranged in concentric rows, which confer the scaffold a typical surface fibrous texture (FIG. 2(b)). That is, the fibrous texture is produced on the scaffold surface by the dendritic planes which are stacked as schematized in the FIG. 4(a) and which emerge up to the surface as the lines extending in the direction of upper right to lower left on the surface of the scaffold in FIG. 2(b). In this sense, every plane corresponds to a “fiber” on the surface. This peculiar arrangement is nothing but the consequence of the circular symmetry impressed by mold inner walls, which are responsible for triggering the heterogeneous nucleation of the solvent.

The structure as a whole can be assumed as the stack of parallel planar fibers intimately in contact, each one representing an array of dendritic units co-penetrating each other (FIG. 4(a)). The single pore unit is evidenced in the SEM image of FIG. 4(b) and schematized by the simplified model of FIG. 4(c), as the superposition of a number of polymeric lamellar layers (three in FIG. 4(c)), whose amount depends on the scaffold thickness.

Each section of the pore unit is constituted by three main lamellae which depart from the stingray sectioned central channel. Two of the lamellae elongate from the stingray wings in the row direction (y direction), towards the first neighboring pore unit, while the third one (generally shorter) departs from the stingray tail in the radial direction (x direction). Referring to FIG. 4(c), the parameters d and D were defined as the diameter of the minor pole of the channel section (stingray tail) and the channel cross-sectional largest dimension (stingray wing-to-wing aperture), respectively. The diagonal side channels observed in FIG. 2(a) correspond to the inter-space between two adjacent lamellae in the vertical direction z. This spacing is about 20 μm, thus comparable with d, but can decrease to 15-18 μm in case of pronounced lamellar bending. However, the effective space available for cell accommodation along the side branches of the dendritic unit is about 50 μm, corresponding to the length of the side lamellae in the x direction FIG. 4(c).

The observation of the cross sections of the dTIPS samples evidenced that, at the micron scale, the scaffold fracture tends to occur in correspondence of the necking point of the stingray tail, thus leaving the tail channel well exposed on the section planes. The tail channel diameter corresponds to d and can be easily measured. The tail channel can also be observed in the non directional samples, as the fracture front proceeds through ordered domains. For this reason, the diameter d was assumed as a control parameter for establishing the dependence of the pore size on the quenching temperature. Its value was determined for each sample by the average of those measured by SEM micrographs visual analysis. However, it must be noted that the main channel aperture D, which is always about three times larger than d, is the effective parameter that must be considered for cell accommodation evaluation.

Table 1 shows the porosity and average pore size values found for the dTIPS scaffolds, together with the values for TIPS scaffolds for sake of comparison. The dTIPS-prepared constructs showed the same pore size d of those resulting from isotropic thermal processing at the same TPS, confirming the strict dependence of pore size on thermal conditions. The scaffold thickness ranged from 600 μm to over 1 mm, depending on the solution volumes used.

TABLE 1

| Synthesis | | | | Overall | Interconnected |
|---|---|---|---|---|---|
| method | T_{PS} | \|ΔT\| | d | porosity | porosity |
| (TIPS) | (° C.) | (° C.) | (μm) | (%) | (%) |
| non- | +4 | ~6 | 150 | 77 | 68 |
| directional | | | | | |
| non- | −20 | ~30 | 45 | 86 | 33 |
| directional | | | | | |
| non- | −40 | ~50 | 25 | 89 | 86 |
| directional | | | | | |
| non- | −80 | ~90 | 13 | 94 | 90 |
| directioual | | | | | |
| directional | −40 | ~50 | 25 | 93 | 91 |

Tps: phase separation temperature
ΔT: under-cooling amounts

One can observe that both the overall and interconnected porosity values observed in the case of scaffold prepared by directional TIPS at −40° C. (93% and 91%, respectively) were larger than those related to isotropic TIPS performed at the same temperature, while being comparable to those measured for the TIPS sample prepared at −80° C., despite the large temperature difference. This finding, as well as the high degree of interconnectivity exhibited by the dTIPS scaffolds, must be correlated with the ordered configuration, and can be explained by taking into account the different mechanisms behind solvent shaping for the investigated regimes. In the case of non-directional isotropic TIPS, PLLA solution undergoes a non-coordinated dioxane crystallite formation. This results in a short range ordering/packing that ends in randomly oriented domains joined together by amorphous transition regions where the expelled polymer accumulates. This leads to a non-co-continuous solvent phase and thus to the presence of closed porosity, after dioxane dissolution. On the other side, the aligning effect imposed by the uni-axial thermal gradient in dTIPS is such to cause the maximization of the crystallite density and thus of the matrix void content. Moreover, as solidification proceeds, branches from different crystallites can eventually interpenetrate, leading to highly interconnected empty spaces after dioxane removal.

### Example 2

(1) Cell Deep Seeding and Cell Culturing on dTIPS 3D Scaffold:

If not clearly stated differently, all the biological validation tests were performed on the PLLA scaffolds fabricated by dTIPS at −40° C. Before cell seeding, scaffolds undergo a preliminary vacuum treatment aimed to replace the air trapped within the porous cavities with cell-free culture medium, in order to permit homogeneous cell culturing in the entire scaffold volume. The 14 mm diameter round scaffolds were EtOH rinsed, vacuum dried, and UV sterilized for 5 minutes per each side. Each sample was then placed into a 10 mL plastic syringe having same diameter of the cylindrical scaffold, locked to the bottom with a polytetrafluoroethylene (PTFE) cylinder as shown in FIG. 5a. The syringe was partially immersed in a culture medium containing petri-dish (not shown) from where the culture medium fluid was forced to pass through the scaffold porosity by repeated aspiration and subsequent air expulsion as shown in FIGS. 5b and 5c. At the end of the treatment, the scaffold was observed to sink into the culture medium. The flooded scaffold was moved to a plastic dish, covered by fresh culture medium, and left equilibrating in incubator for 24 h at 38° C. The medium was replaced with new cell loaded serum in concentration of 8.0·10⁴ cells·mL⁻¹ through a second aspiration/expulsion cycle, analogous to the previous one, which brought dispersed cells deep into the scaffold as shown in FIGS. 5a and 5c. Seeded constructs were gently removed from the syringe and transferred to a 24-well culture dish and kept in incubator at 37° C., 5% CO₂ for 18 h, for attachment screening purposes, or up to 14 days for proliferation evaluation, with culture medium being replaced twice a week.

### (2) Validation

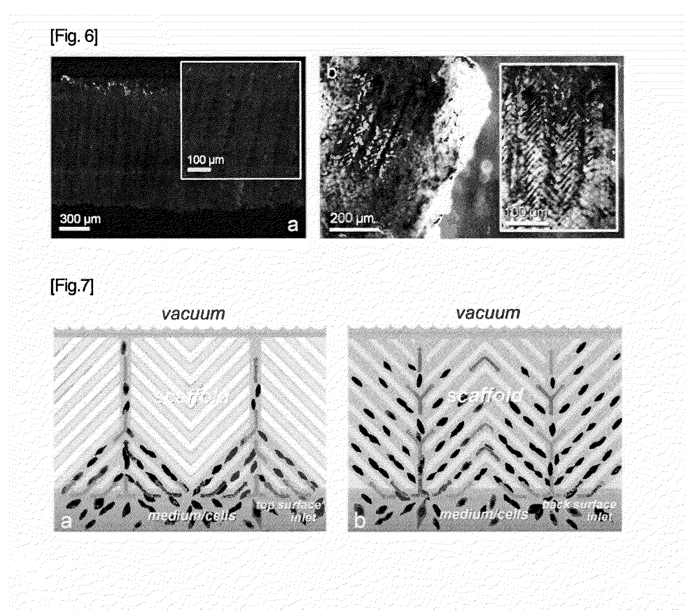

FIG. 6 shows the cross sectional view of a pre-treated scaffold of about 1 mm in thickness before cell culturing (FIG. 6(a)) and after 18 h of bone marrow derived cells (MSC) culturing (FIG. 6(b)). Images were acquired under optical microscope, after hematoxylin/eosin cell staining. The cross sections were obtained by gently ripping the scaffold along its diameter. A sharp edge blade was subsequently used to reduce the thickness of the exposed surface down to about 500˜800 μm, to allow the light to pass through the structure and the cell-colonized fracture surface to be observed. As shown in FIG. 6(a), the vacuum-forced flooding treatment did not alter nor damage the scaffold pore architecture. On the contrary, it allowed having an already medium-permeated scaffold, which has the advantage to smoothly drive cell accommodation in the support interior. Under these conditions, it is expected that cells can quickly reach every site accessible in the porous scaffold since they can migrate through the same medium in the whole scaffold thickness, instead of experiencing preferential attachment to the unaltered PLLA hydrophobic surfaces during infiltration.

As previously stated, cell seeding was performed under vacuum. The cell seeding outcome is shown in FIG. 6(b). Cells have significantly colonized the interior of the sample (about 1 mm thick) moving along the dendritic pathways. These findings confirm (i) the effectiveness of the vacuum based seeding method, (ii) the absence of damages both to the scaffold and cells, and (iii) the effectiveness of the interconnected pores to allow cell diffusion into the whole scaffold thickness even up to 1 mm. Cells can access the inner part of the scaffold directly from the axial pores or by sliding through the surface ends of the lateral branches.

### (3) Mechanism and Powerpoints of the Here Developed Vacuum-Aided Deep Seeding Method

As previously mentioned, the here proposed seeding methods was setup specifically for the dTIPS scaffolds of the present invention. In fact, this method exploits the scaffold peculiar geometry which is non tortuous and characterized by high mass transfers throughout the porous structure. However, as shown schematically in FIG. 7, the infiltration process can be very different if performed from the bottom or the top scaffold surface, given the oriented fishbone structure of the pores. Referring to the fishbone cross section, the two possible mechanisms proposed are visually described in FIG. 7. When cells are forced within the scaffold from the top side, the surface terminal lamellae act like a multifunneled structure, whose effect is to convoy the cell flux mainly into the vertical channels shown in FIGS. 7a and 7b. When cells are infiltrated from the bottom side as shown in FIG. 7(b), the reverse-funnel configuration facilitates cell migration from the backbone channels to the side branches, leading to a more uniform cell distribution within the construct. Therefore, to improve an even cell distribution, the scaffold was positioned with its back face adjacent to the syringe inlet, both during the above-described flooding treatment and cell seeding steps.

### Example 3

(1) 2D dTIPS Scaffold Smart Cut Fabrication:

As already stated, the present invention also provides a method of fabricating planar thin (20-70 μm) scaffolds having oriented dendrite-like porosity, otherwise unachieved so far by dTIPS. This method is a top-down approach starting from the 3D dTIPS scaffold. Thin 2D scaffold were obtained as follows: the 3D dTIPS scaffold was positioned in a plastic syringe and locked by mean of PTFE rings. A 30% sucrose/PBS solution was prepared and stored in a 15 mL plastic tube, and used as embedding solution. Alternatively, a 2% bovine derived gelatin in H₂O dissolved 0.1% in PBS, was used as embedding agent. Alternatively, other embedding solutions can be prepared, using materials and concentrations stated in the first paragraph of (2) Validation of <Example 2>. 6 mL of embedding agent were aspired into the syringe and forced to flow through the scaffold. The embedding solution is expelled and additional 6 mL are aspired again. The syringe outlet is sealed and the scaffold+embedding agent undergo intense vacuum treatment by pulling the syringe piston back and forth. The process is complete when more than 90% of the scaffold volume is sunk in the solution. The ebedded scaffold was placed on a metal dipper in proper orientation and immerged in a dewar in which hexane and dry ice are contained. The frozen sample is covered with OCT (Optimal Cutting Temperature) compound and left solidify. The embedded scaffold embedded in OCT is removed from the freezing bath and mounted on a cryostat chuck at a temperature ranging from −20 to −30° C. Particular attention must be put in choosing the scaffold orientation on the cryostat chuck. The scaffold is mounted in order to exhibit the fishbone-like cross section orientation parallel to the cutting plane. In this case, we were able to produce 60 μm thin polymer slices, each one corresponding to a single dendrite plane, as defined in FIG. 4(a). The cut thin sections were collected using a glass cover slip and kept at −80° C. prior to further use.

### (2) Cell Seeding and Cell Culture of the 2D Scaffolds:

The glass cover slip bearing the 2D scaffold were sterilized under UV light, 5 min each side, and then moved to a plastic dish. 40 μL of cell loaded serum in concentration of 8.0·10⁴ cells·mL⁻¹ were dropped on the single scaffold, so that the drop did not spread outside the scaffold itself. The plastic dish is moved to incubator for 30 min for allowing cell attachment, after which the scaffold is covered by fresh culture medium, and left culturing at 37° C., 5% CO₂. After confluent proto-tissues were detected on the 2D scaffold, the cultured scaffolds were gently lift with forceps and stacked one on another, to constitute a 3D thick construct made of alternation of cell layers and biopolymer. Seeded constructs were analyzed by immuno-fluorescence microscopy upon cell staining. Checkpoints were 24 hours, for attachment screening purposes, or up to 14 days for proliferation evaluation, with culture medium being replaced twice a week.

### (3) Validation

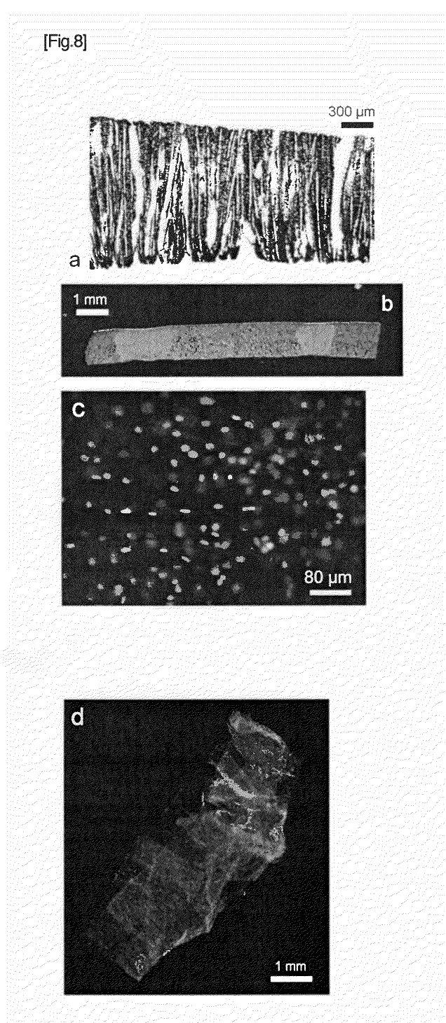

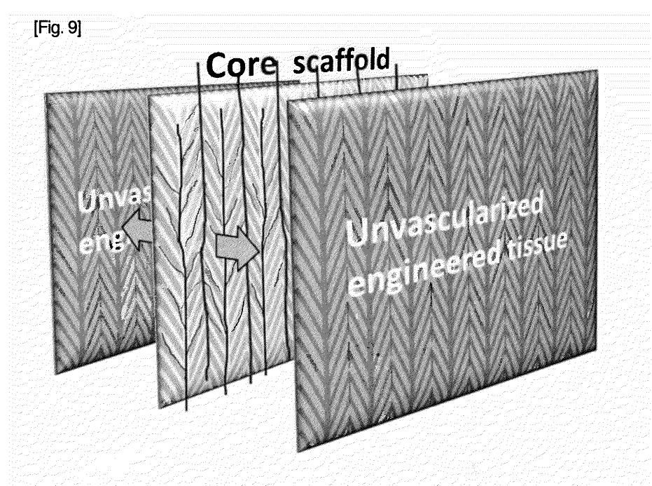

FIGS. 8(a) and (b) refer to a sucrose solution coated thin dTIPS 2D scaffold prior to cell seed, at high and low optical microscope magnification, respectively. One can observe that the peculiar ordered dendritic structure was preserved during the material processing, accounting for the effectiveness and reproducibility of the smart cut slicing method. FIG. 8(c) shows the 2D scaffold colonization by MSC, after 6 DIV (Days In Vitro). The image is acquired in fluorescence mode, and reveals a massive, specific cell adhesion and growth over the coated scaffold. This evidence testifies not only the non toxicity of the engineered materials, but also their ability to efficiently support cell culturing in a viable manner. Finally, FIG. 8(d) shows the constitution of a confluent proto-tissue over a bilayered dTIPS scaffold, i.e., made by superposition of two 60 μm thick section previously cultured with MSC for 15 DIV. The image was acquired after in fluorescence mode upon nuclei staining with DAPI.

Apparatus and methods we used in the above examples are as follows.

### (a) A Cooling Apparatus:

As a cooling unit, we used a cold finger (Julabo FT109; Italy). Sectional view and whole view of “a cold finger” setup is shown in FIG. 1.

### (b) Porosity Measurements:

The weight of each sample Ws was first measured using an analytical balance to get the polymer effective volume (i.e., the volume actually occupied by the polymer) Vp:

Vp=Ws/ρₒ (1)

being ρₒ the measured density of the purchased polymer, equal to 1.27 g cm⁻³. The scaffolds were initially immersed into water. PLLA hydrophobicity and scaffold capillary forces (caused by sub −100 μm pore size) make it impossible for water to penetrate into the structure. The up thrust, or buoyancy received by the sample once immersed in the fluid is proportional to Vₜₒₜ, i.e., the volume occupied by the polymer and the total air (i.e., scaffold's total porosity):

$$\begin{matrix}{UT}_{tot} = {V}_{tot} {ρ}^{′} \\ = {UT}^{′} + {W}_{s}\end{matrix} \qquad ( 2 )$$

wherein UTₜₒₜ is the buoyancy the scaffold receives from the liquid in which it is immersed (i.e., water in this case), ρ′ is 1.0 g cm⁻³ which is the mass density of the liquid, or water, and UT′ is the net buoyancy as measured.

The total porosity εₜₒₜ₋ₐ is defined as below:

εₜₒₜ₋ₐ=[(Vₜₒₜ−Vₚ)Nₜₒₜ]100 (3)

Since the effective volume of polymer Vₚ is obtained by using equation (1) and the total volume of the scaffold is obtained based on the value of the net buoyant force measured by using equation (2), the total porosity εₜₒₜ₋ₐ can be calculated by using equation (3).

The interconnected porosity value ε_{IC}, that is, the ratio of all the holes that can be reached outside of the scaffold by passing any existing holes to the entire scaffold can be obtained as follows:

The weight of the scaffold is measured in ethanol bath by immersing a sample in ethanol for two hours for having ethanol completely penetrate into it. The difference between expected weight Wₑ and the actually measured weight Wₐ is obtained, where the expected weight We is the weight expected by assuming that ethanol has completely penetrated into all the holes in the scaffold. The expected weight Wₑ and the actually measured weight Wₐ are represented respectively as below:

$$\begin{matrix}\begin{matrix}We = ( the weight of the polymer ) + \\ ( the weight of the ethanol in all the holes ) - \\ ( the buoyant force exerted on the entire scaffold ) . \\ = [ \begin{matrix}( the weight of the polymer ) - \\ ( \begin{matrix}buoyant force exerted on the \\ effective volume of the polymer\end{matrix} )\end{matrix} ] + \\ [ \begin{matrix}( the weight of tthe ethanol in all the holes ) - \\ ( the buoyant force exerted on all the holes )\end{matrix} ] \\ = ( {W}_{s} - {V}_{p} {ρ}^{′} ) + 0\end{matrix} & \\ \begin{matrix}{W}_{a} = ( the weight of the polymer ) + \\ ( the weight of the ethanol in the interconnected holes ) - \\ ( the buoyant force exerted on the entire scaffold ) \\ = [ \begin{matrix}( the weight of the polymer ) \\ ( \begin{matrix}the buoyant force exerted on the \\ effective volume of the polymer\end{matrix} )\end{matrix} ] + \\ [ \begin{matrix}( \begin{matrix}the weight of the ethanol in \\ the interconnected holes\end{matrix} ) - \\ ( \begin{matrix}the buoyant force exerted \\ on the interconnected holes\end{matrix} )\end{matrix} ] + \\ [ 0 - ( the buoyant force exerted on the closed holes ) ] \\ = ( {W}_{s} - {V}_{p} {ρ}^{′} ) + 0 - {V}_{CL} {ρ}^{′}\end{matrix} & \end{matrix}$$

wherein ρ′ is the mass density of ethanol, i.e., 0.8 g cm⁻³, and V_{CL} is the volume of the all the closed holes.

Thus, the difference therebetween amounts to

$$\begin{matrix}{W}_{e} - {W}_{a} = ( {W}_{s} - {V}_{p} {ρ}^{′} ) - [ ( {W}_{s} - {V}_{p} {ρ}^{′} ) - {V}_{CL} {ρ}^{′} ] \\ = {V}_{CL} {ρ}^{′}\end{matrix}$$

That is, this is the buoyant force exerted on all the closed holes. With the above result, the closed porosity is obtained as below:

$$\begin{matrix}{ɛ}_{CL} = {V}_{CL} / {V}_{tot} \\ = ( {W}_{e} - {W}_{a} ) / ( {ρ}^{′} {V}_{tot} ) \\ = [ ( {W}_{s} - {V}_{p} {ρ}^{′} ) - {W}_{a} ] / ( {ρ}^{′} {V}_{tot} )\end{matrix}$$

Since all the parameters in the above equation are known, the closed porosity value ε_{CL} can be obtained. Thus, the interconnected porosity value ε_{IC} can also be obtained.

### (c) Scanning Electron Microscopy (SEM):

Scaffold morphology was mainly determined by optical and scanning electron microscopy (SEM) observations. The non-biologically treated scaffolds were mounted on aluminum stubs using adhesive conducting carbon tape and then gold sputtered for 2 min (EMITECH K500X, Ashford, England), prior to be observed in a field-emission scanning electron microscope (FE-SEM, LEO SUPRAl250, Oberkochen, Germany) operating at acceleration voltages of 8˜10 kV. Once cell seeded, PLLA scaffolds underwent cell fixation and dehydration prior to SEM investigation. Briefly, cells were fixed by covering the scaffold with 2.5% glutaraldehyde (GA) in self made phosphate buffer (PB) 0.1 M at pH 7.2, for 2 h and then washed several times in PB. A post-fixation treatment followed in 1% osmium tetraoxide (OsO4) in PB at room temperature overnight. The construct was further washed in PB and subsequently kept in PB-EtOH solution at increasing EtOH concentrations from 30% to 100%. Once dried in air, the sample was finally ready for metallization.

### (d) Mechanical Characterization:

Scaffold mechanical properties were investigated by means of tensile testing. The main round sample was cut into 0.5 cm×2 cm stripes, being the 2 cm side oriented either along or perpendicular to the sample radius in the case of the dTIPS scaffolds. The stripes were mounted on a Thumler Z3 (Germany) tensile tester set at a speed rate of 2 mm/min. For each test, the tensile strength was normalized to the average sectional area of the sample under measurement, obtained by coupling thickness measurements and SEM observations.

### INDUSTRIAL APPLICABILITY

As explained above in detail, the present invention provides highly porous solid materials made of biodegradable polymer and their fabrication method. According to the invention, we can easily produce the above highly porous biodegradable scaffolds and successfully culture them in vitro. The invented highly porous biodegradable solid materials (or scaffolds) has a new morphology (pore/channel network) which is characterized by the ordered repetition of parallel closely-packed dendrite-like cavities, resembling the vascular patterns. The original seeding method specifically developed for these scaffolds allows instant and efficient cellular colonization of the construct, which is unachievable in scaffolds with differently organized porosities. The engineered scaffolds of the present invention are expected to serve for application aimed to vascular net and angiogenesis.

### PRIOR ART DOCUMENTS

### Non-patent Documents

[Non-patent Document 1] F. Yang et al.: Biomaterials 2006, vol. 27, 4923.
[Non-patent Document 2] Ch. Schugens et al.: Polymer 1996, vol. 37, 1027.
[Non-patent Document 3] X. Hu et al.: Biomaterials 2008, vol. 29, 3128.
[Non-patent Document 4] P. X. Ma et al.: J. Biomed. Mater. Res. 2001, vol. 56, 469.
[Non-patent Document 5] G. Wei et al.: Biomaterials 2004, vol. 25, 4749.
[Non-patent Document 6] D. K Gilding et al.: Polymer 1979, vol. 20, 1459-84.
[Non-patent Document 7] J. Kohn, R. Langer. Bioresorbable and Bioerodible Materials. In: B. D. Ratner, A. S. Hoffman, F. J. Schoen, J. E. Lemons (eds). Biomaterials science: an introduction to materials in medicine, New York, Academic Press (1996), 64-72.
[Non-patent Document 8] W. P. Werschler: Dermatol. Ther. 2007, Suppl 1, 16-9.
[Non-patent Document 9] Orthopedic Biomaterials, The World Market, 2nd Edition, Kalorama Information (2007).

1. A highly porous solid material made of biodegradable polymer wherein a pore network of said material icomprises ordered repetition of parallel, closely-packed dendritic-like cavities.

2. The material according to claim 1, wherein said biodegradable polymer is poly-L-lactic acid.

3. The material according to claim 1, wherein an alignment direction of main pores of said parallel, closely-packed dendrite-like cavities extends perpendicular from one surface of said material to an opposite surface to said one surface.

4. The material according to claim 3, wherein a diameter of said main pores of said dendric-like cavities is 70±10 μm and a diameter of side branches of said dendritic-like cavities is 45±5 μm and wherein a distance between adjacent ones of said main pores is 80±20 μm.

5. The material according to claim 4, wherein said side branches depart from said main pores at an angle of 45° to 70° with respect to axes of said main pores.

6. A method of fabricating a 3D highly-porous solid material made of biodegradable polymer wherein a pore network of said material comprises an ordered repetition of parallel, closely-packed dendritic-like cavities, the method comprises the steps of:
(i) dissolving biodegradable polymer in dioxane at a defined concentration;
(ii) mounting a vessel containing a solution of said dioxane with said biodegradable polymer dissolved on a metal plate thermally driven by a cooling unit to a defined temperature below 10° C., and then keeping said vessel on said plate until said solution undergoes complete phase separation;
(iii) immersing solids obtained from said phase separation into an alcohol aqueous solution to leach out dioxane.

7. The method according to claim 6, wherein said vessel is a poly-tetrafluoroethylene mold.

8. The method according to claim 6, wherein shape of said vessel is cylindrical.

9. The method according to claim 6, wherein said biodegradable polymer is poly-L-lactic acid.

10. The method according to claim 9, wherein poly-lactic acid concentration in step (i) is in between 2 wt % and 15 wt %.

11. The method according to claim 6, wherein said defined temperature is a constant temperature in between −20 and −80° C.

12. The method according to claim 6, wherein a bottom of said vessel is cooled so that a thermal gradient may be applied to said solution perpendicular to said bottom.

13. A vacuum-assisted method of seeding cells within a scaffold made of the material according to claim 1 comprising the steps of
(a) positioning said scaffold in a first syringe;
(b) applying a vacuum treatment to said scaffold for replacing air trapped within said cavities with cell-free culture medium;
(c) positioning in a second syringe said scaffold flooded with said cell-free culture medium;
(d) applying a vacuum treatment to said scaffold positioned in said second syringe for replacing said cell-free culture medium with fresh cell-loaded medium;
(e) placing said scaffold containing said cell-loaded medium in a culture well with covered with fresh culture medium; and
(f) keeping said scaffold in said culture well in a incubator.

14. The method according to claim 13, wherein at least one of said first and second syringes is made of plastic.

15. The method according to claim 13, wherein said scaffold is locked with PTFE rings in at least one of said first and second syringes.

16. A method of fabricating a 2D highly-porous solid material made of biodegradable polymer having a pore network constituted by an ordered repetition of parallel, closely-packed dendrite-like cavities from a 3D scaffold made of the material according to claim 1, said pore network being arranged so that, on cutting said 3D scaffold along a predetermined plane, said dendritic-like cavities appear in a fishbone-like structure on a cross-section, the method comprising the steps of
(a) embedding said 3D scaffold with a specific agent by using a vacuum treatment;
(b) freezing said embedded scaffold;
(c) placing said frozen scaffold on a cryostat chuck in a proper orientation;
(d) smart cut by means of cryostat microtome along the selected scaffold section; and
(e) slicing said 3D scaffold in parallel to said predetermined plane.

17. The method according to claim 16, wherein said agent is sucrose in ultrapure water solution.

18. The method according to claim 17, wherein concentration of sucrose ranges from 1% to 40% in H₂O.

19. The method according to claim 16, wherein said agent is bovine derived gelatin in ultrapure water.

20. The method according to claim 19, wherein concentration of bovine derived gelatin ranges from 1% to 40% in H₂O.

21. The method according to claim 16, wherein said agent is bovine derived gelatin in PBS.

22. The method according to claim 21, wherein the bovine derived gelatin solution is used at a concentration of 0.1% to 30% in PBS.
